(19) United States
(12) Patent Application Publication
(10) Pub. No.: US 2024/0027479 A1
(43) Pub. Date: Jan. 25, 2024

(54) A Comprehensive Breath Test that Confirms Recent Use of Inhaled Cannabis within the Impairment Window

(71) Applicant: RCU Labs, Inc., Roseville, CA (US)

(72) Inventors: Michael W DeGregorio, Granite Bay, CA (US); Gregory T Wurz, Lincoln, CA (US); Chiao-Jung Kao, Granite Bay, CA (US); Edward J Montoya, Sacramento, CA (US)

(73) Assignee: RCU Labs, Inc., Roseville, CA (US)

(21) Appl. No.: 18/265,541

(22) PCT Filed: Oct. 26, 2021
(86) PCT No.: PCT/US2021/056591

§ 371 Date: Jun. 6, 2023

Related U.S. Application Data
(63) Continuation-in-part of application No. 17/048,737, filed on Oct. 19, 2020.
(60) Provisional application No. 63/128,547, filed on Dec. 21, 2020.
(60) Provisional application No. 62/661,280, filed on Apr. 23, 2018.

(51) Int. Cl. G01N 33/94; A61B 5/097; A61B 5/08

(52) U.S. Cl. CPC G01N 33/948; A61B 5/097; A61B 5/082

(57) ABSTRACT

The present invention provides a method for determining recent use of cannabis, including recent use within the impairment window, following administration through inhalation, the method comprising the collection of samples of exhaled breath and whole blood separated by known intervals of time, analyzing them using appropriate analytical methods, and then calculating pharmacologic parameters associated with recent cannabis use. In particular embodiments, the method is used by employers in the routine monitoring of workplace drug policy compliance among employees and in workplace accident investigations. In some embodiments, the method is utilized by law enforcement personnel to gath-er evidence in driving under the influence investigations.

### CROSS-REFERENCES TO RELATED APPLICATIONS

This application claims priority to U.S. Provisional Patent Application No. 63/128,547, filed Dec. 21, 2020, the disclosure of which is hereby incorporated by reference in its entirety for all purposes.

### BACKGROUND OF THE INVENTION

Cannabis (Cannabis spp), used by human beings for thousands of years, is a complex plant that contains over 100 cannabinoids and hundreds of other chemical compounds such as terpenes and flavonoids [ElSohly M A. et al., Prog Chem Org Nat Prod 103:1-36 (2017)]. The main psychoactive cannabinoid found in cannabis is Δ⁹-tetrahydrocannabinol (Δ⁹-THC), which exerts its activity by binding to endogenous cannabinoid receptors known as CB₁ and CB₂ [Maccarrone M. et al., Trends Pharmacol Sci 36:277-96 (2015)]. Other common cannabinoids found in cannabis include cannabidiol (CBD), cannabigerol (CBG), cannabinol (CBN), cannabichromene (CBC), and Δ⁹-tetrahydrocannabivarin (Δ⁹-THCV). Within the cannabis plant, CBD, CBG, Δ⁹-THCV, and Δ⁹-THC exist predominantly in their carboxylic acid forms, which undergo decarboxylation upon sufficient heating. To become psychoactive, Δ⁹-tetrahydrocannabinolic acid A (Δ⁹-THCA), which is the natural, carboxylic acid form of Δ⁹-THC, requires decarboxylation [Grotenhermen F., Clin Pharmacokinet 42:327-60 (2003)].

Despite several different available testing methods for assessing cannabis use, the ability to effectively establish recent use within the impairment window, generally accepted to be approximately three hours [Hartman R L. & Huestis M A., Clin Chem 59(3):478-92 (2013); Couper F J. & Logan B K., National Highway Traffic Safety Administration DOT HS 809 725 (2014)], has remained elusive. As a result, inappropriate interpretation of test results by employers, for example, can lead to job applicants not getting hired, or to the wrongful termination of current, non-federal employees who are using cannabis lawfully and responsibly in jurisdictions that allow such use, and where employees are not serving in safety-sensitive positions. As of late 2021, recreational cannabis had been legalized in 19 U.S. states plus Washington, D.C. Some of these states, including Nevada, New Jersey, and New York, have begun to enact laws that ban employ-ment discrimination against current or prospective employees who engage in legal, off-duty recreational cannabis use. Ironically, Colorado, one of the first states to legalize recreational cannabis, provides little legal protection of off-duty cannabis use by employees. At the same time, studies have suggested that the legalization of recreational cannabis has been associated with an increase in fatal traffic collisions [Aydelotte J D. et al., Accid Anal Prev 132:105284 (2019); Santaella-Tenorio J. et al., JAMA Intern Med 180(8):1061-8 (2020)]. Based on data collected from 2007-2018, a recently published study showed that in U.S. states where recreational cannabis is legal, there has been a 15% increase in fatal collisions and a 16% increase in associated deaths, changes that were sustained beyond the first year following legalization [Windle S B. et al., CMAJ Open 9(1): E233-E241 (2021)]. Clearly, a new type of test that can identify recent cannabis use within the impairment window is needed to help bring an end to cannabis discrimination practices in the workplace while also accurately detecting inappropriate cannabis use, e.g., driving under the influence (DUI), to protect public safety.

Although some U.S. states, e.g., Colorado and Washington, have enacted legal Δ⁹-THC limits for cannabis DUI, no definitive correlation between the degree of impairment and specific blood levels of Δ⁹-THC has been established [Brubacher J R. et al., Addiction 114:1616-26 (2019); Hartman R L. et al., Accid Anal Prev 92:219-29 (2016); Logan B. et al., An Evaluation of Data from Drivers Arrested for Driving Under the Influence in Relation to Per Se Limits for Cannabis, AAA Foundation for Traffic Safety (May 2016); Etue K K. et al., Report from the Impaired Driving Safety Commission, Michigan State Police (March 2019)]. The state of California and other U.S. states where cannabis has been legalized presently rely on specially trained police officers known as drug recognition experts to make a determination of DUI due to cannabis or other drugs. Physical indicators that have been strongly correlated with cannabis impairment include the condition of the eyes (e.g., reddening of conjunctiva, bloodshot, and watery) and their physiological response (e.g., horizontal gaze nystagmus) [Hartman R L. et al., Accid Anal Prev 92:219-29 (2016); Porath A J. et al., Traffic Inj Prev 20:255-63 (2019); Bosker W M. et al., Psychopharmacology 223:439-46 (2012)]. Law enforcement agencies desperately need an objective means of assessing impairment associated with recent cannabis use.

Similar to the situation on the roadways, working under the influence of drugs such as cannabis is a potential workplace safety hazard both to the individual and surrounding personnel, especially when impaired individuals are operating heavy machinery or motor vehicles, or performing critical work duties, for example, firefighters or emergency medical personnel. With the expanding legalization of cannabis for medical and recreational use in the United States and abroad, testing for recent cannabis use, defined as use within approximately three hours for inhalation routes of administration (e.g., smoking and vaping) or within approximately eight hours for edibles [Huestis M A. et al., Clin Chem 51:2289-95 (2005); Couper F. et al., In: Drugs and Human Performance Fact Sheets, National Highway Traffic Safety Administration Report DOT HS 809 725:1-100 (2004); Vandrey R. et al., J Anal Toxicol 41:83-99 (2017)], has become a major safety and liability issue. Because a correlation between Δ⁹-THC and metabolite levels in the blood and recent use or impairment has never been established, current testing methods that rely solely on determining Δ⁹-THC and metabolite concentrations in blood [Biecheler M B. et al., Traffic Inj Prev 9:11-21 (2008); Karschner E L. et al., Drug Test Anal 8:682-9 (2016); Wong A. et al., Drug Alcohol Depend 133:763-7 (2013)], urine [Huestis M A. et al., Trends Mol Med 24:156-72 (2018); Lowe R H., Drug Alcohol Depend 105:24-32 (2009)], saliva [Vandrey R. et al., J Anal Toxicol 41:83-99 (2017); Lowe R H., Drug Alcohol Depend 105:24-32 (2009); Marsot A. et al., J Pharm Pharm Sci 19:411-22 (2016); Swortwood M J. et al., Drug Test Anal 9:905-15 (2017); Moore C. et al., Forensic Sci Int 212:227-30 (2011)], or breath [Lynch K L. et al., Clin Chem 65:1171-79 (2019)] are inconclusive when investigating cannabis use in the event of workplace incidents or suspected DUI.

In humans, 11-hydroxy-A 9-THC (11-OH-A 9-THC) is the main intermediate metabolite of A 9-THC [Matsunaga T. et al., Life Sci 56:2089-95 (1995)]. The most psychoactive of the Δ⁹-THC metabolites, 11-OH-A 9-THC has been shown to be equipotent to Δ⁹-THC in humans [Perez-Reyes M. et al., Science 177:633-5 (1972)]. This metabolite is subsequently eliminated in the feces or oxidized to the inactive metabolite 11-nor-9-carboxy-A 9-THC (Δ⁹-THC—COOH), which is then eliminated in the urine [Grotenhermen F., Clin Pharmacokinet 42:327-60 (2003)]. Less common Δ⁹-THC metabolites are formed by side chain hydroxylation at the 1′, 2′, 3′ or 4′ position, 8α- and 8β-hydroxylation, and 9α,10α- and 9β,10β-epoxidation [Dinis-Oliveira R J., Drug Metab Rev 48:80-7 (2016)]. The metabolism of Δ⁹-THC is summarized in FIG. 1.

The complex nature of cannabinoid pharmacokinetics and pharmacodynamics calls for a new approach for effectively determining recent use of cannabis within the impairment window. We sought to develop a multiple-parameter test based on pharmacological changes in Δ⁹-THC and its metabolites in blood over time. This approach requires the collection of two samples separated by a known time interval, which is a critical difference compared to current blood testing methods that rely on the collection of a single sample. The two-sample strategy makes possible the detection of Δ⁹-THC in its distribution phase where the half-life is very short [Moeller M R. et al., J Forensic Sci 37:969-83 (1992); Wall M E. et al., Clin Pharmacol Ther 34:352-63 (1983)], which occurs only within the first few hours after smoking, and for the evaluation of how Δ⁹-THC metabolite levels in blood are changing with time relative to Δ⁹-THC. A liquid chromatography high-resolution mass spectrometry (LC-HRMS) bioanalytical method for the quantification of cannabinoids in microsamples (50 μL) of whole blood was developed and validated for this purpose [DeGregorio M W. et al., J AOAC Int 103:725-35 (2020)]. We hypothesized that the incorporation of a second testing matrix, i.e., exhaled breath, using the same two-sample strategy to detect kinetic changes in breath cannabinoid levels, could strengthen the results of the blood-based test, which by itself cannot be used to establish impairment, prevent false positive test results, and definitively establish whether a subject is in the impairment window following the use of cannabis through inhalation, i.e. smoking or vaping.

While it has been known for over 30 years that Δ⁹-THC can be detected in exhaled breath [Manolis A. et al., Clin Biochem 16:229-33 (1983)], only relatively recently has this matrix been explored as a potential means of establishing recent cannabis use within the impairment window [Himes S K. et al., Clin Chem 59:1780-9 (2013); Coucke L. et al., Clin Biochem 49:1072-7 (2016)]. Exhaled breath testing for recent use based on Δ⁹-THC alone is predicated on a short period of detection within the impairment window. A study by Himes et al. suggested that Δ⁹-THC is generally detectable in breath for only about two hours after smoking even in chronic users [Himes S K. et al., Clin Chem 59:1780-9 (2013)], but more recent studies have shown that Δ⁹-THC remains detectable in breath for up to several days since last use [Lynch K L. et al., Clin Chem 65:1171-9 (2019); 011a P. et al., Cannabis Cannabinoid Res 5(1):99-104 (2020)]. This is a major finding because no meaningful correlation has yet been established between impairment and the levels of Δ⁹-THC in any matrix tested to date. Because the leading technologies for breath-based testing for recent cannabis use rely solely on detection of Δ⁹-THC [Lynch K L. et al., Clin Chem 65(9):1171-9 (2019); Mirzaei H. et al., J Breath Res 14(3):034002 (2020)], there is a real potential for false positive test results due to the presence of Δ⁹-THC in breath outside of the impairment window.

Given the increasing acceptance of cannabis for both medicinal and recreational use in the United States and internationally, and the current lack of an effective method of discriminating recent use of cannabis from past use, there is a need in the art for methods of determining recent cannabis use so that impaired individuals can be more accurately identified without penalizing those who test positive for prior cannabis use but who are not impaired.

### BRIEF SUMMARY OF THE INVENTION

In some aspects the present invention provides a method for determining recent use of cannabis through inhalation (e.g., smoking or vaping) within the impairment window (i.e., within three hours of use) in human subjects, the method comprising: (1) the collection of one or more (e.g., two) samples of exhaled breath separated by a known time interval (e.g., five minutes) utilizing a device containing a filter (e.g., an electrostatic polymer filter) designed to capture exhaled breath aerosols containing nonvolatile drug molecules and retain them for later laboratory analysis; and (2) the collection of one or more (e.g., two) samples of whole blood separated by a known time interval (e.g., 20 minutes) utilizing a device that automatically collects and stores capillary blood for later laboratory analysis. The results of the laboratory analysis of the blood samples are used to compute the probability, based on a statistical model, that a subject has recently used cannabis by inhalation. The blood-based model is based on six or more parameters (e.g., eight parameters) that have been associated with the recent use of cannabis. The higher the number of parameters for which a subject is positive, the higher the probability of recent cannabis use. The results of the laboratory analysis of the breath samples are used to identify parameters (e.g., 12 parameters) that have been associated with recent cannabis use through inhalation within the impairment window. In the event that a single blood sample is collected alongside two breath samples, the blood sample is used as a confirmatory test. If the breath/blood Δ⁹-THC concentration or intensity ratio is ≥2, this confirms recent cannabis use through inhalation within the impairment window.

In other aspects, the present invention provides a method for determining recent use of cannabis through inhalation (e.g., smoking or vaping) within the peak impairment window (i.e., within one hour of use) in human subjects, the method comprising the collection of two (2) samples of exhaled breath utilizing a device containing a filter (e.g., an electrostatic polymer filter) designed to capture exhaled breath aerosols containing nonvolatile drug molecules and retain them for later laboratory analysis, and one (1) sample of whole blood utilizing a device that automatically collects and stores capillary blood for later laboratory analysis. The results of the laboratory analysis of the breath samples are used to identify parameters (e.g., 12 parameters) that have been associated with recent cannabis use through inhalation within the peak impairment window. The blood sample is used as a confirmatory test to verify recent use within the peak impairment window. If the results show a short Δ⁹-THC half-life (<60 minutes) and at least one other recent use parameter in breath, and the breath/blood Δ⁹-THC concentration or intensity ratio is ≥2, this constitutes a positive test result.

In particular aspects, the method is used by law enforcement personnel to collect evidence in DUI investigations, the method comprising the collection of two (2) exhaled breath samples approximately five minutes apart using devices equipped with electrostatic polymer filters that capture exhaled breath aerosols containing nonvolatile drug molecules and retain them for later analysis, and the collection of two (2) blood samples approximately 20 minutes apart using devices that automatically collect and store capillary blood for later analysis. The samples are then subjected to laboratory analysis for determination of Δ⁹-THC, Δ⁹-THC metabolites, and other cannabinoids. Following the analyses, blood sample data are compared to a list of eight pharmacologic blood-based parameters associated with recent use of cannabis, and the breath sample data are compared to a list of 12 pharmacologic breath-based parameters associated with recent use of cannabis. A positive result in four of the eight blood-based parameters indicates a 95% probability of recent use, and a positive result in five or more of the eight blood-based parameters indicates a 99% probability of recent cannabis use. A positive result in four or more of the eight blood-based parameters combined with a positive result in the breath samples (e.g., Δ⁹-THC half-life <60 minutes, Δ⁹-THC breath/blood concentration or intensity ratio ≥2, and at least one other recent use parameter) indicates that the subject is within the period of peak impairment (i.e., within one hour after smoking or vaping) following cannabis use through inhalation. Additional parameters may be used as they are identified to strengthen the statistical power of the model.

In some embodiments, the method is used by employers in the routine monitoring of workplace drug policy compliance among employees, the method comprising the collection of one (1) breath sample utilizing a device containing a filter (e.g., an electrostatic polymer filter) designed to capture exhaled breath aerosols containing nonvolatile drug molecules and retain them for later laboratory analysis, and the collection of one (1) blood sample using a device that automatically collects and stores capillary blood for later analysis. Other blood collection techniques, e.g., the use of lancing devices similar to those used by diabetics for routine blood glucose monitoring, may be used. The breath and blood samples are then subjected to laboratory analysis for the determination of Δ⁹-THC, Δ⁹-THC metabolites, and other cannabinoids. Following analysis, the breath data are compared to a list of six (6) pharmacologic parameters associated with recent cannabis use by inhalation within the peak impairment window. The presence of CBC and/or Δ⁹-THCV in breath, combined with a breath/blood Δ⁹-THC concentration or intensity ratio ≥2, indicates that the test subject is positive for recent use within the peak impairment window. Additional breath-based parameters of recent use may be employed as they are identified to strengthen the method.

In other embodiments, the method is used by employers in the routine monitoring of workplace drug policy among employees, the method comprising the collection of two (2) breath samples utilizing devices containing a filter (e.g., an electrostatic polymer filter) designed to capture exhaled breath aerosols containing nonvolatile drug molecules and retain them for later laboratory analysis, and one (1) blood sample using a device that automatically collects and stores capillary blood for later analysis. Other blood collection techniques, e.g., the use of lancing devices similar to those used by diabetics for routine blood glucose monitoring, may be used. The breath and blood samples are then subjected to laboratory analysis for the determination of Δ⁹-THC, Δ⁹-THC metabolites, and other cannabinoids. Following analysis, the breath data are compared to a list of twelve (12) pharmacologic parameters associated with recent cannabis use by inhalation within the peak impairment window. A Δ⁹-THC half-life of <60 minutes combined with positivity for at least one other breath-based parameter of recent use and a breath/blood Δ⁹-THC concentration or intensity ratio ≥2 indicates that the subject is positive for recent use of cannabis through inhalation within the impairment window. Additional parameters may be used as they are identified to strengthen the model.

In particular embodiments, the method is used by employers in the investigation of workplace accidents, the method comprising the collection of two (2) exhaled breath samples and two (2) blood samples. Exhaled breath samples are collected approximately five (5) minutes apart using devices equipped with electrostatic polymer filters that capture exhaled breath aerosols containing nonvolatile drug molecules and retain them for later analysis. The blood samples are collected approximately 20 minutes apart using devices that automatically collect and store capillary blood for later analysis. Other blood collection techniques, e.g., the use of lancing devices similar to those used by diabetics for routine blood glucose monitoring, may be used. Following laboratory analysis for determination of Δ⁹-THC, Δ⁹-THC metabolites, and other cannabinoids, the breath sample data are compared to a list of 12 pharmacologic breath-based parameters associated with recent use of cannabis, and the blood sample data are compared to a list of eight (8) pharmacologic blood-based parameters associated with recent use of cannabis. A positive result in four (4) or more of the eight (8) blood-based parameters combined with a positive result in the exhaled breath samples (e.g., Δ⁹-THC half-life <60 minutes, Δ⁹-THC breath/blood concentration or intensity ratio ≥2, and positivity for at least one other recent use parameter) indicates that the subject is within the period of peak impairment (i.e., within one hour after smoking or vaping) following cannabis use through inhalation. A negative breath test result combined with a positive result in four (4) or more of the eight (8) blood-based parameters indicates a >95% probability of recent cannabis use only (within 8-12 hours). Additional parameters may be used to strengthen the statistical power of the model.

In some embodiments, the method is used to determine recent use by inhalation of drugs of abuse other than cannabis (e.g., amphetamines, opiates, and cocaine), the method comprising collection of at least one (1) exhaled breath sample utilizing a device containing a filter (e.g., an electrostatic polymer filter) designed to capture exhaled breath aerosols containing nonvolatile drug molecules and retain them for later laboratory analysis, and at least one (1) blood sample using an automatic capillary blood collection device as described or other acceptable means (e.g., lancets). The samples are then subjected to laboratory analysis for the determination of key drug compounds and metabolites of interest. Following analysis, the data are compared to a list of six or more pharmacologic parameters (e.g., eight parameters) associated with the recent use of the compound(s) of interest. A positive result in four of the eight parameters indicates a 95% probability of recent use, and a positive result in five or more of the eight parameters indicates a 99% probability of recent use of the compound(s) of interest. Additional parameters may be used as they are identified to strengthen the statistical power of the model.

In particular embodiments, the method is used by law enforcement personnel and employers to collect evidence in DUI and workplace accident investigations, respectively, involving inhaled drugs of abuse other than cannabis, the method comprising the collection of two (2) exhaled breath samples utilizing a device containing an electrostatic polymer filter designed to capture exhaled breath aerosols containing nonvolatile drug molecules and retain them for later laboratory analysis, and two (2) whole blood samples approximately 20 minutes apart using automatic capillary blood collection devices as described or other acceptable means (e.g., lancets), which are then subjected to laboratory analysis for multiple drugs of abuse (e.g., amphetamines, opiates, and cocaine). Following analysis, the data are compared to a list of eight (8) pharmacologic parameters associated with the recent use of the compound(s) of interest. A positive result for four of the eight parameters indicates a 95% probability of recent use by inhalation, while a positive result for five or more of the eight parameters indicates a 99% probability of recent use of the compound(s) of interest. Additional parameters may be used as they are identified to strengthen the statistical power of the model. Fewer parameters may also be used as appropriate.

Other objects, features, and advantages of the present invention will be apparent to one of skill in the art from the following detailed description and figures.

### BRIEF DESCRIPTION OF THE DRAWINGS

FIG. 1. Metabolism of Δ⁹-THC. The major metabolites of Δ⁹-THC and the metabolic enzymes primarily responsible for their formation are shown. CYP=cytochrome P450; UGT=uridine 5′-diphospho-glucuronosyltransferase; gluc=glucuronic acid.

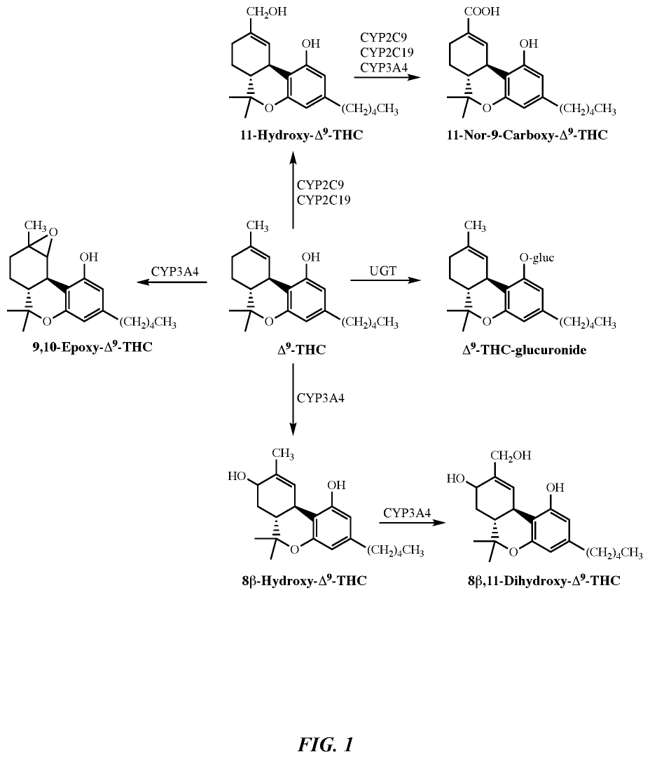

FIG. 2. Self-assessment impairment scale. This scale was used by clinical study subjects to assess their level of impairment prior to and after smoking cannabis.

FIG. 3. Δ⁹-THC concentrations in blood do not correlate with impairment. In a scatter plot, pre-smoking Δ⁹-THC concentrations in blood from 30 clinical subjects are shown compared to a common legal limit for Δ⁹-THC (5 ng/mL; indicated by black horizontal line). Black dashed line indicates the median concentration.

FIG. 4. Impairment windows following cannabis smoking. The window of impairment after smoking cannabis was evaluated in 74 subjects who self-assessed their level of impairment prior to smoking and at various time points post-smoking using a 10-point scale.

FIG. 5. Incidence of horizontal gaze nystagmus before and after smoking cannabis. Overall incidence of nystagmus within the three-hour impairment window (N=34 pre-smoking; N=44 post-smoking; N=43 three hours post-smoking).

FIG. 6. Incidence of horizontal gaze nystagmus before and after smoking cannabis. Incidence of nystagmus within the three-hour impairment window over time (N=34 pre-smoking; N=44 immediately after smoking; N=33 one hour post-smoking; N=10 two hours post-smoking; N=43 three hours post-smoking).

FIG. 7. Average percent maximum impairment pre-smoking up to three hours post-smoking (N=74). Error bars indicate positive standard deviation.

FIG. 8. Breath/blood Δ⁹-THC ratios. Solid black horizontal lines indicate mean values. N=29 (pre-smoking baseline; BL); N=32 (immediately after smoking; 0 min); N=35 (20 min post-smoking; N=24 (60 min post-smoking); N=31 (180 min post-smoking). Zero values (13 at BL and two at 180 min) are not shown due to logarithmic scale. Due to assay quantification limits, three values (two immediately after smoking and one at 60 min post-smoking) were excluded. *(p=0.0015), †(p=0.0108), ‡(p=0.0107), § (p=0.0091) compared to BL (two-tailed t-test with Bonferroni's adjustment for multiple comparisons; α=0.0125).

### DETAILED DESCRIPTION OF THE INVENTION

I. INTRODUCTION

This invention relates to the development of a pharmacologic test based on exhaled breath and blood sampling for detecting recent use of cannabis within the impairment window following consumption of cannabis products through inhalation, e.g., smoking or vaping. The condition and physiological response of the eyes (e.g., horizontal gaze nystagmus) are used as physical indicators of impairment. In blood or dried blood spots, recent use is based on pharmacokinetic information for Δ⁹-THC, its principal metabolites (11-0H-A 9-THC, 11-nor-9-carboxy-A 9-THC, and 8β,11-dihydroxy-A 9-THC), concentration or intensity ratios of these metabolites to Δ⁹-THC, and the ratios between different metabolites. The presence of, and pharmacokinetic information pertaining to, key recent use indicators, including but not limited to CBN, CBC, CBG, Δ⁹-THCV, Δ⁹-THC epoxides, and Δ⁹-THC-glucuronide, will also be used as blood-based evidence of recent use. At least two (2) blood samples are collected to provide the necessary pharmacokinetic information. The second blood sample is ideally collected approximately 20 minutes after collection of the first sample. Breath-based evidence to support recent cannabis use within the impairment window includes, but is not limited to, pharmacokinetic information pertaining to key cannabinoids (e.g., Δ⁹-THC and CBN) and the presence of key indicators of recent use (e.g., CBC, CBG, Δ⁹-THCV) in the breath. At least two (2) breath samples are collected to obtain this information. Breath samples are preferably collected no more than 20 minutes apart, more preferably five minutes apart. Alternatively, it is possible to demonstrate recent cannabis use within the peak impairment window based on a single exhaled breath sample, one (1) exhaled breath and one (1) blood sample, or two (2) exhaled breath samples and one (1) blood sample, as described below.

Should distribution phase kinetics be observed (e.g., a half-life of <60 minutes for Δ⁹-THC or CBN) for key cannabinoids in breath, in addition to a positive test result from blood or dried blood spot samples and physical evidence of impairment, this is definitive evidence that the subject was within the peak impairment window (the first hour after smoking or vaping) when the samples were collected. Physical evidence of impairment gathered during standardized field sobriety testing includes a finding of horizontal gaze nystagmus or other physiological responses (e.g., red, bloodshot, watery eyes) that are indicative of recent cannabis use. In the event that distribution phase kinetics and recent use indicators are not observed in the breath, but the subject receives a positive test result in blood or dried blood spots, this is indicative only of recent cannabis use (within approximately 8-12 hours). In order to prevent false positive test results from breath samples, certain criteria may be applied. For example, to be considered a valid positive test result for recent use within the impairment window, the breath/blood Δ⁹-THC concentration or intensity ratio must be ≥2.

An obvious advantage of this invention is in the law enforcement setting, as there are currently no effective methods for establishing recent cannabis use within the impairment window in DUI investigations. Current methods rely on single measurements of cannabinoids in blood, urine, saliva, or breath, none of which provides sufficient evidence of recent use or impairment due to cannabis. For cases of suspected DUI, it is critical for the law enforcement officer to collect breath samples as quickly as possible because peak impairment occurs within the first hour after smoking or vaping cannabis and evidence dissipates rapidly. Due to the non-invasive nature of breath sampling, no warrant is required, which will facilitate this process. Immediately upon suspicion of DUI, two breath samples are collected approximately three to five minutes apart utilizing single-use breath collection devices. Nonvolatile drug molecules contained within exhaled breath aerosols are captured by the electrostatic polymer filter within the collection device. While a longer time interval (e.g., 20 minutes) can be used if necessary, the shorter time interval between samples helps prevent the loss of rapidly dissipating evidence. If a driver is arrested on suspicion of DUI, blood samples should also be collected as soon as possible to minimize the dissipation of evidence. Blood sampling can be performed concomitantly with breath sampling, but this is not required. At least two (2) blood samples are collected approximately 20 minutes apart using automated devices designed to collect capillary blood. Such a device operates in a fashion similar to lancing devices used by diabetics for blood glucose testing; that is, the device will lance a suitable body part (e.g., the upper arm) so that a small quantity of blood (e.g., 250 μL) may be collected and stored within a sealed chamber or collection tube containing anticoagulant. As a convenient alternative, dried blood spots may also be used, and devices are available that are capable of automatically collecting capillary blood and preparing dried blood spots. Lancets for collecting capillary blood are also acceptable, in addition to traditional venipuncture. As soon as possible after collection, all breath and blood samples are shipped to the laboratory for analysis. For convenience, the supplies needed for breath and blood sample collection, identification, and shipping can be combined within a kit designed for law enforcement applications.

Another advantage of this invention is in the setting of workplace drug testing in states where recreational and/or medicinal use of cannabis has been legalized. For employers, the exhaled breath and blood test will better differentiate those employees who have used cannabis recently and may be impaired from employees who have used cannabis in the past, but not recently enough to be impaired. This would protect both the employer, who can identify impaired employees who pose a genuine threat to the business, and the employees, who can avoid unjustified termination as a result of legal and responsible cannabis use. The availability of this invention gives employers a compelling reason to abandon zero-tolerance policies, which are unnecessarily burdensome, in those jurisdictions where such policies have not already been disallowed for many non-public safety related positions through legislation. In a workplace environment, two (2) breath samples are collected approximately five minutes apart, and either a single blood sample or two (2) blood samples collected approximately 20 minutes apart, are taken depending on the desired application. For example, in the event of a workplace accident where maximum evidence to support impairment is needed, two (2) breath and two (2) blood samples are collected. For routine monitoring of employees for drug policy compliance, employers may elect to collect only two (2) breath samples with or without a single blood sample, which can be used to establish recent use of cannabis by inhalation within the peak impairment window when sufficient evidence is observed (one or more breath-based parameters of recent use combined with a breath/blood Δ⁹-THC ratio ≥2). Blood may be collected via venipuncture, automated capillary blood collection devices, or lancet. As soon as possible after collection, all breath and blood samples are shipped to the laboratory for analysis. For convenience, the supplies needed for breath and blood sample collection, identification, and shipping can be combined within kits customized for workplace applications.

Blood and breath samples will be analyzed in a laboratory for the determination of various cannabinoids and metabolites including, but not limited to, Δ⁹-THC, 11-OH-A 9-THC, 11-nor-9-carboxy-Δ⁹-THC, 8β,11-dihydroxy-Δ⁹-THC, CBN, CBG, CBC, Δ⁹-THCV, CBGA, Δ⁹-THC glucuronide, and Δ⁹-THC epoxides using validated analytical methods; for example high-performance liquid chromatography (HPLC), gas chromatography tandem mass spectrometry (GC-MS/MS), liquid chromatography tandem mass spectrometry (LC-MS/MS), or LC or GC high-resolution mass spectrometry (HRMS), as appropriate. Other drugs of abuse and their various metabolites will be analyzed utilizing these methods. Once the concentrations or relative intensities of these compounds have been determined, the various pharmacologic parameters will be calculated so that a determination of recent cannabis use within the impairment window can be made.

The breath and blood-based test method can also be used for DUI and non-DUI investigations related to inhaled drugs of abuse other than cannabis including, but not limited to, illegal synthetic cannabinoids, methamphetamines, and cocaine. The type of filter contained within the breath collection devices utilized in testing for recent cannabis use is already known to capture multiple different drugs of abuse in exhaled breath, drugs and metabolites of which that are readily detectable in blood.

The application of this invention is not restricted to just a two-point blood and two-point breath analysis. Three, four, or more blood and breath samples can be collected to further strengthen the predictive accuracy of the model. When testing only for recent use within the peak impairment window (e.g., in a workplace setting), only a single breath sample may provide sufficient evidence that the subject was within the peak impairment window following the use of cannabis through inhalation (i.e., smoking or vaping), as mentioned above. One breath and one blood sample may likewise be sufficient; for example, if the breath/blood Δ⁹-THC concentration or intensity ratio is ≥2, and there is at least one other recent use indicator observed in the breath (e.g., the presence of CBC and/or Δ⁹-THCV), this is sufficient evidence of recent use within the impairment window.

An exhaled breath and blood-based model utilizing multiple recent use parameters will be used to determine whether a test subject recently used cannabis and was within the established impairment window (approximately three hours after use through inhalation). For example, a positive breath test result (short Δ⁹-THC half-life and at least one other recent use indicator) combined with supporting blood evidence (breath/blood Δ⁹-THC concentration or intensity ratio ≥2) indicates recent cannabis use within the impairment window. A negative breath test result and a positive blood test result (e.g., four or more positive parameters out of eight) indicates recent use of cannabis only through inhalation within the last 8-12 hours. When considering blood evidence alone, the greater the number of recent use parameters for which the subject is positive, the higher the statistical probability of recent cannabis use through inhalation. The model is based on the following breath and blood-based parameters that have been associated with recent use of cannabis through inhalation. This list is for illustration purposes only. As additional recent use parameters are identified, they may be added to the model.

A short Δ⁹-THC half-life (less than one hour) in breath, blood, or dried blood spots, indicating distribution phase kinetics, when comparing two test samples.

A short CBN half-life (less than one hour) in breath, blood or dried blood spots, indicating distribution phase kinetics, when comparing two test samples.

A short Δ⁹-tetrahydrocannabinolic acid A (Δ⁹-THCA) half-life (less than one hour) in breath, indicating distribution phase kinetics, when comparing two test samples.

A short 11-OH-Δ⁹-THC half-life (less than one hour) in blood or dried blood spots, indicating distribution phase kinetics, when comparing two test samples.

A short CBG half-life (less than one hour) in breath, indicating distribution phase kinetics, when comparing two test samples.

A short CBC half-life (less than one hour) in breath, indicating distribution phase kinetics, when comparing two test samples.

A short Δ⁹-THCV half-life (less than one hour) in breath, indicating distribution phase kinetics, when comparing two test samples.

A ratio of 11-nor-9-carboxy-Δ⁹-THC to Δ⁹-THC in blood or dried blood spots that is increasing at least 25% when comparing two test samples.

A ratio of 11-nor-9-carboxy-Δ⁹-THC to CBN in blood or dried blood spots that is increasing at least 25% when comparing two test samples.

A ratio of 11-OH-Δ⁹-THC to Δ⁹-THC in blood or dried blood spots that is increasing at least 25% when comparing two test samples.

A ratio of 8β,11-dihydroxy-Δ⁹-THC to Δ⁹-THC in blood or dried blood spots that is increasing at least 25% when comparing two test samples.

A ratio of 11-nor-9-carboxy-Δ⁹-THC to 11-OH-Δ⁹-THC in blood or dried blood spots that is increasing at least 25% when comparing two test samples.

The presence of CBG in breath.

The presence of CBC in breath.

The presence of Δ⁹-THCV in breath.

The presence of Δ⁹-THC-glucuronide in whole blood or dried blood spots.

The presence of Δ⁹-THC epoxides in whole blood or dried blood spots.

A short CBGA half-life (less than one hour) in breath, indicating distribution phase kinetics, when comparing two test samples.

The presence of CBGA in breath.

A breath/blood Δ⁹-THC concentration or intensity ratio that is ≥2.

In summary, a pharmacologic model based on a combination of exhaled breath and blood testing has been developed for the assessment of recent cannabis use within the impairment window. As a theoretical example, if breath and blood samples from a subject show a Δ⁹-THC half-life less than one hour, a breath/blood Δ⁹-THC concentration or intensity ratio ≥2, and are positive for least one other breath-based recent use parameter, this is sufficient evidence of recent use within the peak impairment window (within one hour after use). A subject with a negative breath test but who's blood samples are positive for at least four (4) recent use parameters out of a total of eight (8) has a >95% probability of having used cannabis recently (within the last 8-12 hours) through inhalation. It is also possible to determine recent cannabis use through inhalation within the impairment window based on breath samples only. As a non-limiting example, if exhaled breath samples from a subject show a Δ⁹-THC half-life <60 minutes in addition to at least one other recent use indicator, e.g., the presence of Δ⁹-THCV, this is sufficient evidence of recent cannabis use within the peak impairment window.

II. DEFINITIONS

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a parameter” includes a plurality of such parameters, and reference to “the metabolite” includes reference to one or more metabolites known to those skilled in the art, and so forth.

The terms “subject,” “patient,” or “individual” are used herein interchangeably. The term “sample” includes whole blood, capillary blood, plasma, serum, breath, oral fluid, and urine.

As used herein, the term “consumption” includes smoking, vaping, oral administration, sublingual administration, topical contact, and administration as a suppository. One skilled in the art will know of additional methods of administering cannabis and cannabis-derived compounds.

The term “cannabinoid” refers to any member of the broad class of phytocannabinoid compounds derived from the cannabis plant (Cannabis spp.), or their synthetic equivalents, including, but not limited to, Δ⁸-THC, Δ⁹-THC, CBD, CBG, CBC, CBN, Δ⁹-THCV, and any of their associated carboxylic acid forms.

The term “metabolite” includes any of the products resulting from the metabolism of cannabinoids within the body, including, but not limited to, 11-0H-Δ⁹-THC, 11-nor-9-carboxy-Δ⁹-THC, 8β,11-dihydroxy-Δ⁹-THC, Δ⁹-THC-glucuronide, and Δ⁹-THC epoxides.

Δ⁸-THC and Δ⁹-THC refer to, respectively, Δ⁸-tetrahydrocannabinol and Δ⁹-tetrahydrocannabinol.

11-OH-Δ⁹-THC refers to the metabolite 11-hydroxy-Δ⁹-tetrahydrocannabinol.

Δ⁹-THC—COOH refers to the metabolite 11-nor-9-carboxy-Δ⁹-tetrahydrocannabinol.

Δ⁹-THCV refers to the cannabinoid Δ⁹-tetrahydrocannabivarin.

Δ⁹-THCA refers to the cannabinoid Δ⁹-tetrahydrocannabinolic acid A.

CBN refers to the cannabinoid cannabinol.

CBD refers to the cannabinoid cannabidiol.

CBDA refers to the cannabinoid cannabidiolic acid.

CBG refers to the cannabinoid cannabigerol.

CBC refers to the cannabinoid cannabichromene.

CBGA refers to the cannabinoid cannabigerolic acid.

The term “illegal synthetic cannabinoid” refers to any of the illegal cannabinoid-like designer drugs that have been identified, including, but not limited to, Spice, K2, synthetic marijuana, AK-47, Mr. Happy, Scooby Snax, Kush, and Kronic.

The term “recent cannabis use test” refers both to a blood-based test applied to determine whether a subject has used cannabis recently through smoking, vaping, or consumption of edibles, and to the breath- and blood-based test applied to determine whether a subject has used cannabis recently through inhalation (e.g., smoking or vaping) and is within the impairment window.

The term “impairment window” refers to the three-hour time period immediately following the use of cannabis through inhalation (e.g., smoking or vaping).

The terms “peak impairment” and “peak impairment window” refer to the one-hour time period immediately following the use of cannabis through inhalation (e.g., smoking or vaping).

III. DESCRIPTION OF THE METHOD

For exhaled breath-based testing or combined breath and blood-based testing for recent cannabis use within the impairment window, the method underlying this invention relies on specific breath-based parameters of recent use. A subject manifesting a short Δ⁹-THC half-life (<60 minutes) and at least one other breath-based indicator of recent use, e.g., the presence of CBC, is considered positive for recent use of cannabis within the impairment window. If additional evidence is desired or if the subject is manifesting only a short Δ⁹-THC half-life, an optional confirmatory test utilizing a blood sample can be performed. If the breath/blood Δ⁹-THC concentration or intensity ratio is ≥2, this confirms that the subject was within the impairment window after having used cannabis through inhalation.

For blood-based testing, the method underlying this invention relies on the use of a statistical model to derive the probability that a DUI suspect, employee, or other test subject recently used cannabis or some other substance of abuse for which testing is desired. Comprising the model are various parameters that have been associated with the recent use of cannabis or other compounds of interest. Ideally, at least six (6) parameters are used, but more may be added as they are identified to strengthen the statistical power of the model. The greater the number of parameters for which a subject is positive, the higher the statistical probability of recent use and therefore impairment. It may also be possible to determine recent use based on fewer parameters. The subjects' pharmacokinetic and pharmacodynamic data are used to determine whether the particular subject is positive, for which a value of “1” is assigned, or negative, for which a value of “0” is assigned, in each of the parameters. An appropriate statistical test is then applied to determine whether the subjects' average scores are significantly different from the hypothetical average of “0,” which would represent a non-recent user.

As a non-limiting example of the application of the invention for blood-based testing, take eight (8) of the described blood-based pharmacologic parameters that have been associated with recent cannabis use. After determining positivity or negativity for each of these eight parameters, a Student's t-test with two-sample equal variance, two-tailed distribution, and significance level of 0.05 is then applied. The null hypothesis for this test is that there is no meaningful difference between the subject's average score for the eight parameters and that of a hypothetical non-recent user. The result of this test will return the probability (p) that the test subject could have come from a population of non-recent users. A p-value result of <0.05 is considered statistically significant. In the example of eight (8) pharmacologic parameters, a statistically significant result is achieved when the test subject is positive for four (4) or more of the parameters. Other statistical tests may be suitable and may be employed in the evaluation. This model may be adapted to test for the recent use of other drugs of interest, and additional parameters may be added to this model to strengthen its statistical power. It may also be possible to use fewer than eight parameters, e.g., at least one or as many as seven, to determine recent use based on blood evidence.

When considering recent cannabis use and impairment, based on the specific positive test parameters in exhaled breath and blood samples, a test subject may be placed into one of the following three categories: (1) a recent cannabis user who is within the peak impairment window (positive test result in breath and blood); (2) a recent user who is outside of the peak impairment window (positive test result in blood only); or (3) a non-recent user (negative test result in blood and breath). As a non-limiting example, a test subject whose samples show short half-lives for Δ⁹-THC and CBN in breath and blood or dried blood spots, and a short 11-OH-Δ⁹-THC half-life, Δ⁹-THC—COOH/A 9-THC ratio increasing ≥25% in blood or dried blood spots, and Δ⁹-THC-COOH/CBN ratio increasing ≥25% in blood or dried blood spots, combined with a breath/blood Δ⁹-THC concentration or intensity ratio ≥2, has a >99% probability of being a recent user based on blood evidence alone, and is a recent user within the peak impairment window based on combined breath and blood evidence. A software program can be written that automatically computes positivity or negativity in each parameter specified following sample analysis and places the subject into one of the three categories. This program would be applicable to cannabis use through inhalation.

IV. EXAMPLES

The following examples are offered to illustrate, but not to limit, the claimed invention.

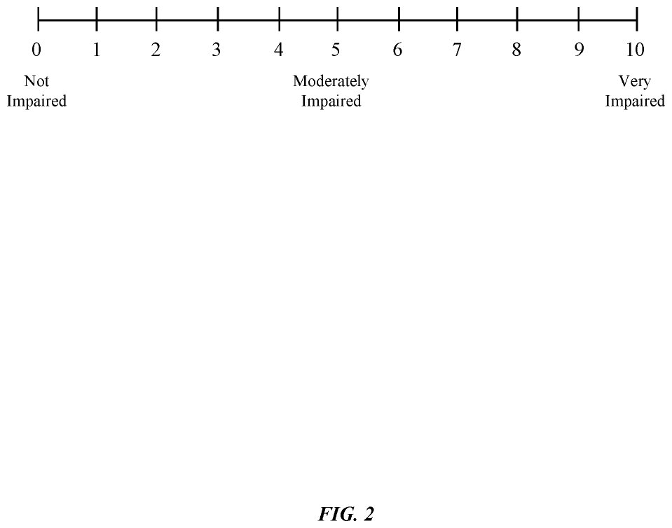

A total of 92 subjects were included in clinical trials designed to evaluate the feasibility of the described method for determining recent cannabis use (blood-based study in 48 subjects) and recent cannabis use and impairment (breath- and blood-based study in 44 subjects). Each subject was given a single cannabis cigarette and instructed to smoke as much of it as possible within a 10-minute period. Cigarettes containing 500 mg of dried cannabis flower were prepared immediately before each smoking session. During the studies, subjects were given a variety of cannabis chemovars to smoke, with Δ⁹-THC content ranging from a low of 8.5% to a high of 28.4%. A wide variety of chemovars was included to account for the variability in Δ⁹-THC potencies available in numerous cannabis retail establishments in the various U.S. states where recreational and/or medicinal cannabis has been legalized. Capillary blood samples (50-100 collected at various time points prior to smoking and up to 200 minutes after smoking, were obtained from a total of 92 subjects using a variety of methods including lancets and two types of automated blood collection devices. Exhaled breath samples were collected from a total of 44 subjects at various time points prior to smoking and up to 240 minutes post-smoking. Breath samples were collected using devices equipped with electrostatic polymer filters and that are designed to collect approximately 20 L of exhaled breath through normal breathing. The time required for breath sample collection was approximately 2-3 minutes. A total of 74 subjects were asked to self-assess their level of impairment before smoking and at each designated sampling time point after smoking based on a scale ranging from 0 (not impaired) to 10 (very impaired) as shown in FIG. 2. As a physical indicator of impairment, 44 subjects were evaluated for horizontal gaze nystagmus.

### Example 1: Δ⁹-THC Blood Levels do not Correlate with Impairment

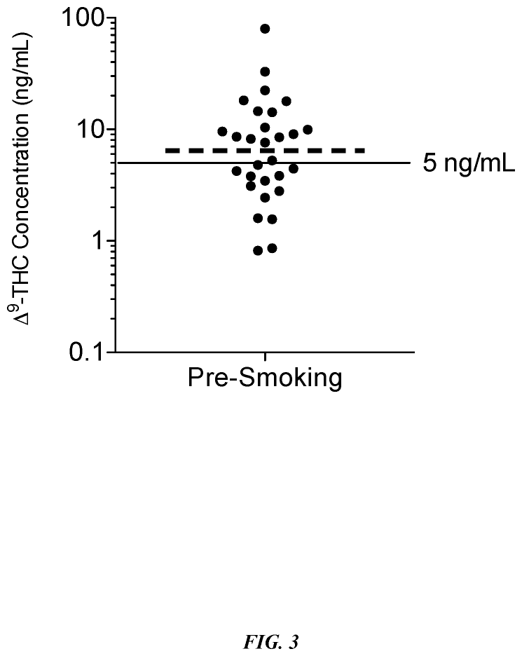

One of the first endpoints investigated during clinical development of the recent use test method was whether there is any relationship between blood Δ⁹-THC levels and impairment. Prior to smoking, Δ⁹-THC blood concentrations were evaluated in a group of 30 subjects before and up to 200 minutes after smoking a 500-mg cannabis cigarette and compared to a common legal limit (5 ng/mL) for establishing driver impairment due to cannabis. As of August 2020, the U.S. states of Illinois, Montana, and Washington were using 5 ng/mL as a legal limit. The results showed that 16 of these subjects had baseline (BL) Δ⁹-THC levels that exceeded 5 ng/mL, while the other 14 had Δ⁹-THC levels ranging between 0 and 4.8 ng/mL, as seen in FIG. 3. The average baseline Δ⁹-THC concentration was 10.5±15 ng/mL (n=30), while the median value (indicated by the dashed line in FIG. 3) was 6.4 ng/mL.

As part of the inclusion criteria for this clinical study, all subjects were instructed to abstain from the use of cannabis products for at least 12 hours, but no more than 24 hours, prior to beginning the study. These subjects were also asked to self-assess their level of impairment on a 10-point scale (FIG. 2) prior to smoking and at all time points after smoking. All 30 subjects reported a zero level of impairment prior to smoking, and no evidence of horizontal gaze nystagmus was observed in any of these subjects prior to smoking. This data is consistent with recently published studies showing no significant correlation between impairment and specific blood concentrations of Δ⁹-THC [Brubacher J R. et al., Addiction 114:1616-26 (2019); Hartman R L. et al., Accid Anal Prev 92:219-29 (2016); Logan B. et al., An Evaluation of Data from Drivers Arrested for Driving Under the Influence in Relation to Per Se Limits for Cannabis, AAA Foundation for Traffic Safety (May 2016); Etue K K. et al., Report from the Impaired Driving Safety Commission, Michigan State Police (March 2019)].

This Example reinforces the fact that a single measure of Δ⁹-THC in the blood or breath by itself is not sufficient to demonstrate recent use of cannabis, with or without impairment. This finding adds to growing body of scientific evidence that Δ⁹-THC levels in blood are not correlated with impairment, and thus the use of legal Δ⁹-THC blood concentration limits is arbitrary and not scientifically supportable.

### Example 2: Window of Impairment Following Cannabis Smoking

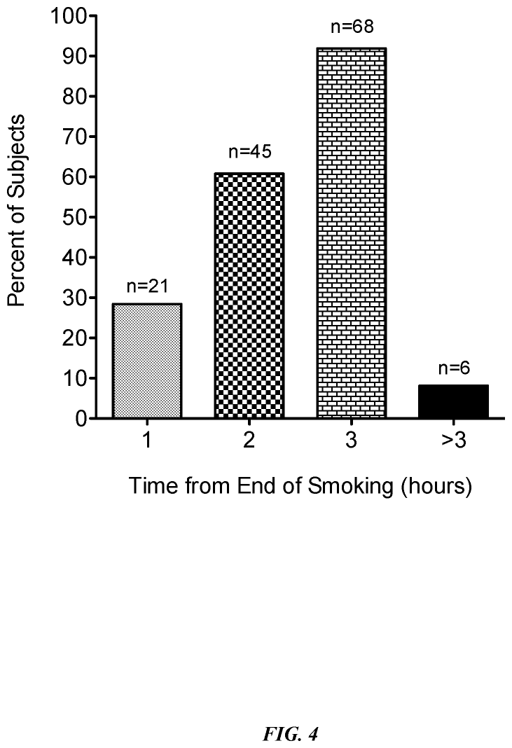

The window of impairment following cannabis smoking was evaluated in 74 subjects who self-assessed their impairment level prior to smoking and at various time points post-smoking using the 10-point scale shown in FIG. 2. Among these subjects, all 74 (100%) reported peak impairment within the first hour after smoking (48 subjects immediately after smoking, 24 subjects at 20 minutes post-smoking, and two subjects at 60 minutes post-smoking) (see Table 1 below). The overall window of impairment was found to be approximately three hours after smoking, at which time 68 of 74 subjects (92%) last reported any impairment. There were six subjects (8%) still reporting some degree of impairment outside this window (see FIG. 4). In these six subjects, the percentage of maximum impairment was an average 23.0±21.7% (range 11.1 to 66.7%). To normalize self-assessed impairment data, the reported impairment levels at each time point for each subject were divided by the maximum reported impairment level for each subject, with the result expressed as a percentage as shown in Table 1 below.

TABLE 1
Percent maximum self-assessed impairment.

| | Percent Maximum Self-Assessed Impairment Post-Smoking (minutes) | | | | | | | | | | |
|---|---|---|---|---|---|---|---|---|---|---|---|
| | Pre- | | | | | | | | | | |
| Subject | Smoking | 0 | 20 | 40 | 60 | 80 | 120 | 140 | 180 | 200 | 240 |
| 1 | 0.0 | 66.7 | 100.0 | —* | 83.3 | 50.0 | 16.7 | 0.0 | 0.0 | 0.0 | — |
| 2 | 0.0 | 33.3 | 100.0 | — | 66.7 | 66.7 | 0.0 | 0.0 | 0.0 | 0.0 | — |
| 3 | 0.0 | 66.7 | 83.3 | — | 100.0 | 100.0 | 83.3 | 66.7 | 33.3 | 0.0 | — |
| 4 | 0.0 | 100.0 | 84.6 | — | 46.2 | 23.1 | 7.7 | 7.7 | 0.0 | 0.0 | — |
| 5 | 0.0 | 60.0 | 100.0 | — | 60.0 | 20.0 | 0.0 | 0.0 | 0.0 | 0.0 | — |
| 6 | 0.0 | 100.0 | 62.5 | — | 12.5 | 0.0 | 0.0 | 0.0 | 0.0 | 0.0 | — |
| 7 | 0.0 | 100.0 | 83.3 | — | 66.7 | 66.7 | 50.0 | 33.3 | 16.7 | 0.0 | — |
| 8 | 0.0 | 66.7 | 100.0 | — | 66.7 | 66.7 | 33.3 | 0.0 | 0.0 | 0.0 | — |
| 9 | 0.0 | 100.0 | 80.0 | — | 40.0 | 0.0 | 0.0 | 0.0 | 0.0 | 0.0 | — |
| 10 | 0.0 | 100.0 | 62.5 | — | 25.0 | 12.5 | 0.0 | 0.0 | 0.0 | 0.0 | — |
| 11 | 0.0 | 100.0 | 100.0 | — | 66.7 | 50.0 | 33.3 | 0.0 | 0.0 | 0.0 | — |
| 12 | 0.0 | 100.0 | 90.0 | — | 80.0 | 80.0 | 60.0 | 50.0 | 30.0 | 20.0 | — |
| 13 | 0.0 | 100.0 | 80.0 | — | 0.0 | 0.0 | 0.0 | 0.0 | 0.0 | 0.0 | — |
| 14 | 0.0 | 100.0 | 83.3 | — | 16.7 | 16.7 | 16.7 | 0.0 | 0.0 | 0.0 | — |
| 15 | 0.0 | 100.0 | 83.3 | — | 50.0 | 33.3 | 0.0 | 0.0 | 0.0 | 0.0 | — |
| 16 | 0.0 | 100.0 | 66.7 | — | 33.3 | 33.3 | 0.0 | 0.0 | 0.0 | 0.0 | — |
| 17 | 0.0 | 75.0 | 100.0 | — | 25.0 | 0.0 | 0.0 | 0.0 | 0.0 | 0.0 | — |
| 18 | 0.0 | 87.5 | 100.0 | — | 50.0 | 50.0 | 37.5 | 12.5 | 12.5 | 0.0 | — |
| 19 | 0.0 | 66.7 | 100.0 | — | 55.6 | 44.4 | 22.2 | 11.1 | 0.0 | 0.0 | — |
| 20 | 0.0 | 20.0 | 80.0 | — | 100.0 | 80.0 | 60.0 | 40.0 | 20.0 | 0.0 | — |
| 21 | 0.0 | 100.0 | 75.0 | — | 37.5 | 0.0 | 0.0 | 0.0 | 0.0 | 0.0 | — |
| 22 | 0.0 | 100.0 | 100.0 | — | 71.4 | 28.6 | 0.0 | 0.0 | 0.0 | 0.0 | — |
| 23 | 0.0 | 100.0 | 77.8 | — | 22.2 | 11.1 | 0.0 | 0.0 | 0.0 | 0.0 | — |
| 24 | 0.0 | 100.0 | 85.7 | — | 71.4 | 57.1 | 42.9 | 28.6 | 14.3 | 0.0 | — |
| 25 | 0.0 | 100.0 | 60.0 | — | 0.0 | 0.0 | 0.0 | 0.0 | 0.0 | 0.0 | — |
| 26 | 0.0 | 75.0 | 100.0 | — | 50.0 | 25.0 | 0.0 | 0.0 | 0.0 | 0.0 | — |
| 27 | 0.0 | 100.0 | 90.0 | — | 80.0 | 60.0 | 50.0 | 40.0 | 30.0 | 15.0 | — |
| 28 | 0.0 | 60.0 | 100.0 | — | 80.0 | 60.0 | 20.0 | 0.0 | 0.0 | 0.0 | — |
| 29 | 0.0 | 100.0 | 100.0 | — | 0.0 | 0.0 | 0.0 | 0.0 | 0.0 | 0.0 | — |
| 30 | 0.0 | 100.0 | 71.4 | — | 42.9 | 28.6 | 14.3 | 0.0 | 0.0 | 0.0 | — |
| 31 | 0.0 | 100.0 | 47.1 | — | 23.5 | 11.8 | 0.0 | — | 0.0 | — | — |
| 32 | 0.0 | 100.0 | 100.0 | — | 33.3 | 33.3 | 0.0 | — | 0.0 | — | — |
| 33 | 0.0 | 100.0 | 42.9 | — | 14.3 | 0.0 | 0.0 | — | 0.0 | — | — |
| 34 | 0.0 | 100.0 | 75.0 | — | 0.0 | 0.0 | 0.0 | — | 0.0 | — | — |
| 35 | 0.0 | 100.0 | 68.8 | — | 43.8 | 37.5 | 25.0 | — | 18.8 | — | — |
| 36 | 0.0 | 100.0 | 100.0 | — | 85.7 | 71.4 | 42.9 | — | 28.6 | — | 0.0 |
| 37 | 0.0 | 100.0 | 66.7 | — | 33.3 | 33.3 | 0.0 | — | 0.0 | — | 0.0 |
| 38 | 0.0 | 100.0 | 75.0 | — | 50.0 | 25.0 | 12.5 | — | 0.0 | — | 0.0 |
| 39 | 0.0 | 100.0 | 83.3 | — | 50.0 | 16.7 | 0.0 | — | 0.0 | — | 0.0 |
| 40 | 0.0 | 100.0 | 100.0 | — | 75.0 | 50.0 | 25.0 | — | 0.0 | — | 0.0 |
| 41 | 0.0 | 66.7 | 100.0 | 83.3 | 16.7 | — | 0.0 | — | 0.0 | — | — |
| 42 | 0.0 | 100.0 | 90.0 | 70.0 | 40.0 | — | 30.0 | — | 10.0 | — | — |
| 43† | 0.0 | 0.0 | 0.0 | 0.0 | 0.0 | — | 0.0 | — | 0.0 | — | — |
| 44 | 0.0 | 100.0 | 0.0 | 0.0 | 0.0 | — | 0.0 | — | 0.0 | — | — |
| 45 | 0.0 | 100.0 | 100.0 | 50.0 | 0.0 | — | 0.0 | — | 0.0 | — | — |
| 46 | 0.0 | 100.0 | 60.0 | 60.0 | 40.0 | — | 0.0 | — | 0.0 | — | — |
| 47 | 0.0 | 100.0 | 60.0 | 30.0 | 10.0 | — | 10.0 | — | 0.0 | — | — |
| 48 | 0.0 | 100.0 | 80.0 | 40.0 | 20.0 | — | 0.0 | — | 0.0 | — | — |
| 49 | 0.0 | 83.3 | 100.0 | 100.0 | 50.0 | — | 16.7 | — | 0.0 | — | — |
| 50 | 0.0 | 85.7 | 100.0 | 42.9 | 28.6 | — | 0.0 | — | 0.0 | — | — |
| 51 | 0.0 | 100.0 | 80.0 | 60.0 | 40.0 | — | 20.0 | — | 0.0 | — | — |
| 52 | 0.0 | 83.3 | 100.0 | — | — | — | — | — | 66.7 | 66.7 | — |
| 53 | 0.0 | 100.0 | 75.0 | — | — | — | — | — | 25.0 | 0.0 | — |
| 54 | 0.0 | 100.0 | 66.7 | — | — | — | — | — | 16.7 | 0.0 | — |
| 55 | 0.0 | 100.0 | 83.3 | — | — | — | — | — | 0.0 | 0.0 | — |
| 56 | 0.0 | 100.0 | 80.0 | — | — | — | — | — | 0.0 | 0.0 | — |
| 57 | 0.0 | 20.0 | 100.0 | — | — | — | — | — | 20.0 | 0.0 | — |
| 58 | 0.0 | 75.0 | 100.0 | — | — | — | — | — | 50.0 | 0.0 | — |
| 59 | 0.0 | 100.0 | 75.0 | — | — | — | — | — | 37.5 | 0.0 | — |
| 60 | 0.0 | 100.0 | 75.0 | — | — | — | — | — | 37.5 | 12.5 | — |
| 61 | 0.0 | 100.0 | 70.0 | — | — | — | — | — | 20.0 | 0.0 | — |
| 62 | 0.0 | 100.0 | 100.0 | — | 75.0 | 62.5 | — | — | 0.0 | 0.0 | — |
| 63 | 0.0 | 100.0 | 77.8 | — | 55.6 | 44.4 | — | — | 22.2 | 11.1 | — |
| 64 | 0.0 | 100.0 | 87.5 | — | 62.5 | 37.5 | — | — | 37.5 | 0.0 | — |
| 65 | 0.0 | 100.0 | 87.5 | — | 62.5 | 50.0 | — | — | 12.5 | 0.0 | — |
| 66 | 0.0 | — | 100.0 | 87.5 | 87.5 | — | — | — | 37.5 | 0.0 | — |
| 67 | 0.0 | — | 100.0 | 75.0 | 50.0 | — | — | — | 25.0 | 0.0 | — |
| 68 | 0.0 | — | 100.0 | 70.0 | 40.0 | — | — | — | 0.0 | 0.0 | — |
| 69 | 0.0 | — | 100.0 | 75.0 | 62.5 | — | — | — | 12.5 | 0.0 | — |
| 70 | 0.0 | — | 100.0 | 80.0 | 70.0 | — | — | — | 0.0 | 0.0 | — |
| 71 | 0.0 | — | 100.0 | 40.0 | 0.0 | — | — | — | 0.0 | 0.0 | — |
| 72 | 0.0 | — | 100.0 | 87.5 | 87.5 | — | — | — | 50.0 | 0.0 | — |
| 73 | 0.0 | — | 100.0 | 88.9 | 77.8 | — | — | — | 33.3 | 0.0 | — |
| 74 | 0.0 | — | 100.0 | 62.5 | 50.0 | — | — | — | 25.0 | 12.5 | — |

*Dashes indicate subjects were not sampled at these time points
†Subject failed to complete the self-assessment form

This Example demonstrates that the window of impairment is approximately three hours after smoking cannabis, in agreement with published research, which validates the subjects' self-assessments of impairment. The Example further demonstrates that the window of peak impairment is approximately one hour after smoking cannabis.

### Example 3: Physical Assessment of Impairment: Horizontal Gaze Nystagmus

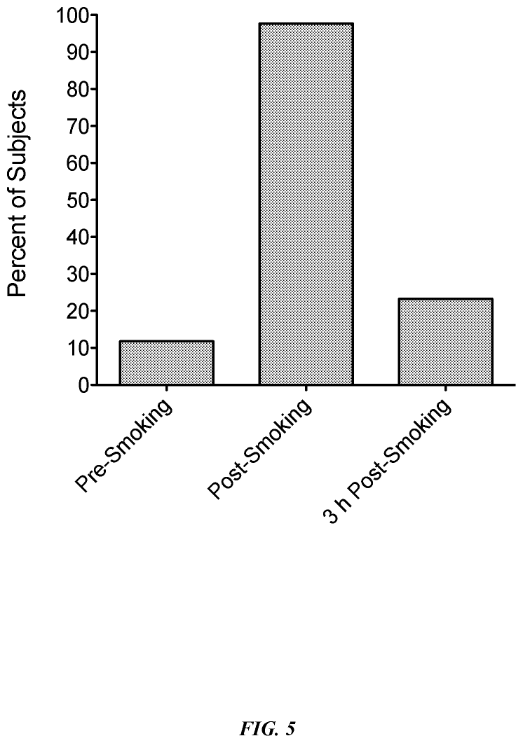

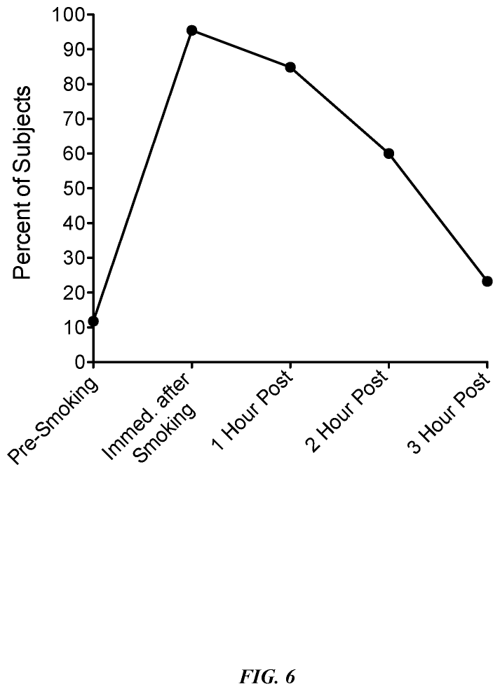

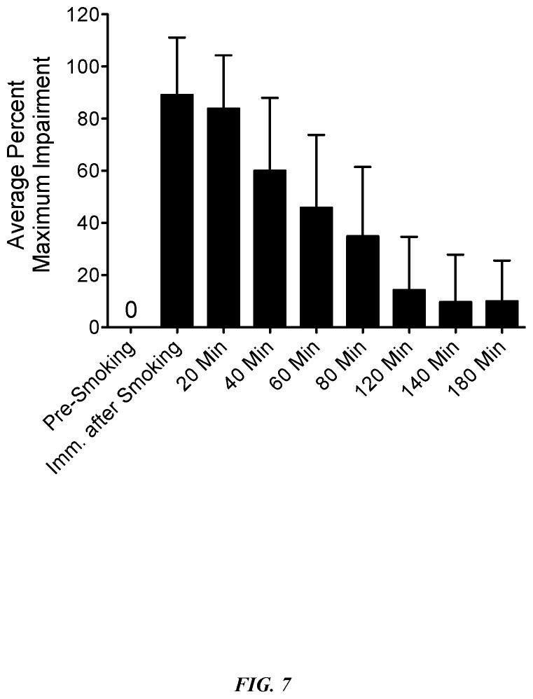

Horizontal gaze nystagmus (HGN) was assessed in 44 subjects. Nystagmus, both horizontal and vertical, refers to the involuntary jerking of the eyes as they gaze up or down. Someone experiencing nystagmus is unaware of its occurrence. Horizontal gaze nystagmus was evaluated prior to smoking cannabis and at various time points up to three hours post-smoking. The results showed that 43 of the 44 subjects (98%) exhibited HGN after smoking cannabis within the three-hour impairment window (FIG. 5). Within the first 20 minutes after smoking, 42 of the 44 subjects (95.5%) exhibited HGN (FIG. 6). These findings corresponded to the self-assessed impairment data, which showed that 97% of subjects (72/74) reported their peak level of impairment within the first 20 minutes after smoking. At three hours post-smoking, the incidence of HGN had fallen to about 23% (10/43 subjects), compared to about 12% prior to smoking (FIG. 6), which also corresponded well to the self-assessed impairment data showing the average percent maximum impairment had fallen to approximately 10% three hours after smoking (FIG. 7).

This Example demonstrates that horizontal gaze nystagmus is an effective physical indicator of impairment following cannabis smoking, and it further verifies the validity of the subject self-assessments of impairment.

### Example 4: Identification of Blood-Based Parameters of Recent Cannabis Use

Blood samples were collected from a total of 92 subjects involved in cannabis smoking studies. After evaluating the pharmacologic data from the first 48 subjects, from whom only blood samples were collected, eight recent use parameters were derived that constitute the blood-based test: (1) Δ⁹-THC half-life <60 minutes; (2) CBN half-life <60 minutes; (3) 11-0H-Δ⁹-THC half-life <60 minutes; (4) an 11-nor-9-carboxy-Δ⁹-THC/Δ⁹-THC ratio that is increasing ≥25%; (5) an 11-nor-9-carboxy-Δ⁹-THC/CBN ratio that is increasing ≥25%; (6) an 11-0H-Δ⁹-THC/Δ⁹-THC ratio that is increasing ≥25%; (7) an 8β,11-dihydroxy-Δ⁹-THC/Δ⁹-THC ratio that is increasing ≥25%; and (8) an 11-nor-9-carboxy-Δ⁹-THC/11-0H-Δ⁹-THC ratio that is increasing ≥25%. Based on the statistical model, a subject would have to be positive for at least four of the eight parameters to receive an overall positive test result in blood. Table 2 below shows percent positivity for each of the eight parameters and overall test positivity for the first 48 subjects from baseline through 200 min post-smoking.

TABLE 2
Blood-based parameters percent positivity with time
after smoking

| | Test Parameters (P) Percent (%) Positive | | | | | | | | |
|---|---|---|---|---|---|---|---|---|---|
| | with Time (n = 25-42) | | | | | | | | |
| | | | | | | | | | % |
| Time | | | | | | | | | Testing |
| (min) | | | | | | | | | Posi- |
| Post- | | | | | | | | | tive |
| Smok- | | | | | | | | | (P < |
| ing | P1* | P2 | P3 | P4 | P5 | P6 | P7 | P8 | 0.05) |
| Base- | 4.0 | 8.0 | 16.0 | 4.0 | 8.0 | 0 | 4.0 | 4.0 | 0 |
| line | | | | | | | | | |
| 20 | 100 | 88.1 | 88.1 | 100 | 88.1 | 81.0 | 88.1 | 88.1 | 100 |
| 60 | 62.9 | 54.3 | 68.6 | 57.1 | 54.3 | 54.3 | 54.3 | 40.0 | 65.7 |
| 140 | 45.9 | 48.6 | 43.2 | 54.1 | 43.2 | 45.9 | 35.1 | 10.8 | 43.2 |
| 200 | 28.9 | 39.5 | 23.7 | 34.2 | 36.8 | 28.9 | 31.6 | 18.4 | 31.6 |

Based on these eight recent use parameters, all 92 subjects were evaluated for recent use prior to smoking (baseline), up to three hours post-smoking, and beyond three hours post-smoking. The results showed that at baseline prior to smoking, 37 out of 37 evaluable subjects (100%) had a negative test result. After smoking, 81 out of 83 evaluable subjects (97.6%) tested positive within the impairment window (up to three hours post-smoking). The two subjects who tested negative based on blood evidence alone were found to be positive based on breath evidence (see Example 5). Outside of the impairment window (>3 hours after smoking), 12 out of 61 evaluable subjects (19.7%) tested positive.

At baseline prior to smoking, all five cannabinoids and Δ⁹-THC metabolites that compose the eight recent use blood-based parameters (Δ⁹-THC, CBN, 11-OH-Δ⁹-THC, 11-nor-9-carboxy-Δ⁹-THC, and 8β,11-dihydroxy-Δ⁹-THC) were detected in the subjects' blood samples. Other cannabinoids and Δ⁹-THC metabolites detected included Δ⁹-THCA, CBD, cannabidiolic acid (CBDA), CBC, CBG, CBGA, Δ⁹-THCV, and 8β-hydroxy-Δ⁹-THC.

This Example shows that the test method based on blood alone is very effective in identifying recent cannabis use, but blood samples alone cannot accurately identify recent use within the impairment window. Additional evidence is needed. The presence of multiple cannabinoids and Δ⁹-THC metabolites in subject blood samples prior to smoking is an important finding because frequent cannabis users will often have detectable levels of multiple cannabinoids in their blood, but this does not constitute evidence of recent cannabis use within the impairment window.

### Example 5: Breath and Blood Combined Test

The positive recent use test results outside of the three-hour impairment window based on blood sampling alone led to the incorporation of a second testing matrix, exhaled breath, to strengthen the results of the test method so that cannabis users who are within the impairment window after smoking can be more accurately identified. For this purpose, a total of 44 additional subjects were evaluated in a smoking study in which both exhaled breath and blood samples were collected and then analyzed by LC-HRMS for cannabinoids and Δ⁹-THC metabolites. As with the 48-subject blood-based study, subjects were given a 500-mg cannabis cigarette to smoke, and breath and blood samples were collected at various time points before and after smoking.

Out of a total of 34 evaluable subjects, 23 (67.6%) had detectable Δ⁹-THC in their breath at baseline prior to smoking (see Table 3 below), which is consistent with recent reports [Lynch K L. et al., Clin Chem 65:1171-9 (2019); 011a P. et al., Cannabis Cannabinoid Res 5(1):99-104 (2020)] showing 100% pre-smoking detection rates of Δ⁹-THC in breath in separate 20-subject and 23-subject studies, respectively. Other cannabinoids detected in exhaled breath prior to smoking included CBN (one subject), CBG (two subjects), and CBGA (four subjects) (see Table 3). Notably, none of the 44 subjects were found to have detectable levels of any Δ⁹-THC metabolites in their breath either before or after smoking.

TABLE 3
Presence of key cannabinoids in exhaled
breath before and after smoking.

| | Percent (%) Positivity | | | | | |
|---|---|---|---|---|---|---|
| Cannabinoid | Baseline | | ≤60 minutes | | >60 minutes | |
| Parameters | (Pre-smoking)* | | after smoking | | after smoking† | |
| Δ⁹-THC | 23/34 | (67.6%) | 40/40 | (100%) | 37/40 | (92.5%) |
| CBN | 1/34 | (2.9%) | 37/40 | (92.5%) | 4/40 | (10.0%) |
| CBC | 0/34 | (0%) | 39/40 | (97.5%) | 0/40 | (0%) |
| CBG | 2/34 | (5.9%) | 37/40 | (92.5%) | 1/40 | (2.5%) |
| CBGA | 4/34 | (11.8%) | 18/40 | (45.0%) | 4/40 | (10.0%) |
| Δ⁹-THCV | 0/34 | (0%) | 36/40 | (90.0%) | 0/40 | (0%) |

*Pre-smoking samples were not collected from 10 subjects.
†No data beyond 60 min post-smoking in 4 subjects.

Table 3 above shows the percent positivity of six cannabinoids prior to smoking, within the first hour post-smoking during peak impairment, and more than one hour post-smoking in subjects' exhaled breath samples. In particular, CBN, CBG, and Δ⁹-THCV all have a much greater incidence in breath during peak impairment (the first hour after smoking) compared to pre-smoking. Interestingly, CBC and Δ⁹-THCV were detected in breath only during the peak impairment window, making these two cannabinoids key indicators of recent cannabis use through inhalation. Stability of the analytes within the breath collection devices was previously established (see Table 4 below).

TABLE 4
Stability of target analytes in the breath collection device

| | Analyte Stability (%) at Room Temperature (20-25° C.) | | | | |
|---|---|---|---|---|---|
| Analyte | Day 3 | Day 7 | Day 10 | Day 14 | Day 30 |
| Δ⁹-THC | 91.3 | 81.2 | 82.9 | 66.2 | 55.6 |
| CBN | 104.5 | 100.0 | 107.3 | 87.9 | 74.4 |
| CBC | 100.6 | 89.1 | 89.9 | 82.2 | 75.6 |
| CBG | 97.5 | 87.6 | 97.8 | 75.0 | 56.5 |
| Δ⁹-THCV | 79.7 | 68.1 | 69.8 | 56.5 | 47.1 |
| CBGA | 99.4 | 110.9 | 110.4 | No data | 114.3 |

After analyzing the breath samples from all 44 subjects, 11 pharmacologic parameters were found to be associated with recent cannabis use based on the multi-point breath sampling strategy, with additional parameters still being investigated. These included the presence of CBN, CBC, CBG, Δ⁹-THCV, and CBGA, and short half-lives (<60 minutes) for Δ⁹-THC, CBN, CBC, CBG, and Δ⁹-THCV (see Table 5 below). Compared to the average Δ⁹-THC half-life measured between 60 and 80 minutes post-smoking (outside of the one-hour peak impairment window post-smoking), the average Δ⁹-THC half-lives measured from immediately after smoking to 20 min post-smoking (p<0.0001), and from 20 to 60 min post-smoking (p=0.0201), were significantly shorter. Pre-smoking, half-lives were calculable for Δ⁹-THC in only three subjects.

TABLE 5
Summary of cannabinoid half-lives in breath post-smoking.

| | Average Half-Life (minutes ± SD) | | | | | | |
|---|---|---|---|---|---|---|---|
| | Minutes Post-Smoking | | | | | | |
| | Pre- | | | | | | |
| Cannabinoid | Smoking | 0-10 | 0-20 | 20-40 | 20-60 | 40-60 | 60-80 |
| Δ⁹-THC | 28.8 ± 28.7 | 4.4 ± 2.2 | 4.2 ± 2.1* | 8.1 ± 2.3 | 12.8 ± 8.8† | 24.4 ± 25.4 | 35.0 ± 41.7 |
| | (N = 3) | (N = 10) | (N = 35) | (N = 9) | (N = 23) | (N = 9) | (N = 6) |
| CBN | —‡ | 3.7 = 2.1 | 3.7 = 3.0 | — | 35.2 | 12.8 | — |
| | | (N = 11) | (N = 20) | | (N = 2) | (N = 1) | |
| CBC | — | 3.9 ± 2.5 | 3.8 ± 2.3 | — | — | — | — |
| | | (N = 10) | (N = 20) | | | | |
| CBG | — | 3.6 ± 2.1 | 3.7 ± 3.1 | — | — | — | — |
| | | (N = 11) | (N = 20) | | | | |
| Δ⁹-THCV | — | 4.2 ± 3.9 | 2.9 ± 0.7 | — | — | — | — |
| | | (N = 9) | (N = 6) | | | | |

*p < 0.0001 compared to 60-80 min;
†p = 0.0201 compared to 60-80 min (two-tailed t-test with Bonferroni's adjustment for multiple comparisons; α = 0.025).
‡Half-life not calculable.

Within the first hour after smoking, which is the period of peak impairment, all 44 subjects (100%) were breath test positive, meaning they all exhibited a short Δ⁹-THC half-life (ranging from 1.0 to 19.1 minutes) and one or more other indicators of recent use in breath. Pre-smoking, all 34 subjects sampled were breath test negative, with two subjects exhibiting only short Δ⁹-THC half-lives. By itself, a short Δ⁹-THC half-life is insufficient evidence of recent use within the impairment window because Δ⁹-THC is commonly seen in the breath of non-recent cannabis users. Additional evidence is needed to confirm recent use.

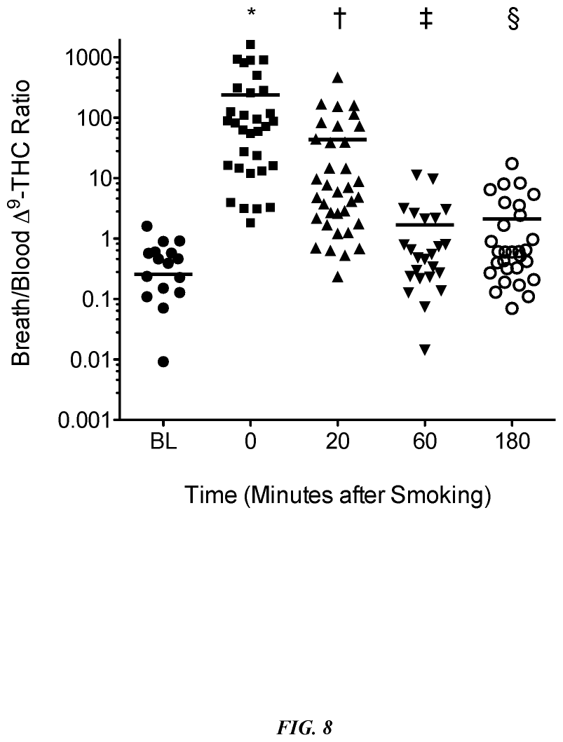

We hypothesized that the incorporation of both exhaled breath and blood into a comprehensive recent cannabis use test would confirm recent use of inhaled cannabis within the impairment window, improving the accuracy of the two-point breath testing method. For this purpose, blood samples were collected from the 44 subjects in addition to breath samples and analyzed for Δ⁹-THC and other cannabinoids. A twelfth recent use parameter that emerged was the breath/blood ratio of Δ⁹-THC, which is a key confirmatory indicator of recent use. This ratio was computed by dividing the Δ⁹-THC peak area ratio to the internal standard in breath by the corresponding peak area ratio in blood. We found that these ratios were >2 in all 44 subjects when assessed immediately after smoking. Compared to the average pre-smoking ratio, the average breath/blood Δ⁹-THC ratios measured immediately after smoking (p=0.0015), 20 minutes post-smoking (p=0.0108), 60 minutes post-smoking (p=0.0107), and 180 minutes post-smoking (p=0.0091) remained significantly greater, as shown in FIG. 8. In the two subjects who exhibited short Δ⁹-THC half-lives in breath pre-smoking, their breath/blood Δ⁹-THC ratios were 0.01 and 0.57, confirming that they had not recently used cannabis within the impairment window.

Within the first hour after smoking, the period of peak impairment, all 44 subjects (100%) were breath and blood test positive, meaning they all exhibited a breath/blood Δ⁹-THC ratio ≥2, in addition to a short Δ⁹-THC half-life and one or more other indicators of recent use in breath. Overall, using the two-point breath and one-point blood test, 0/34 subjects (0%) tested pre-smoking were positive, indicating no false positive results, and 44/44 subjects (100%) were positive inside the three-hour impairment window. This comprehensive test, which incorporates a breath/blood Δ⁹-THC ratio (FIG. 8) and the presence and half-lives of key cannabinoids in breath (Tables 3 and 5), definitively established recent cannabis use within the impairment window.

In some instances, only a single breath and blood sample are needed due to the over-whelming evidence of recent cannabis use in breath. Within the first hour after smoking, all 44 subjects studied with breath sampling showed a breath/blood Δ⁹-THC ratio ≥2 combined with other indicators of recent use. For example, in the two subjects mentioned from Example 4 above who tested negative within the impairment window based on blood sampling alone, very strong evidence of recent use within the peak impairment window based on breath sampling was observed. In each of these subjects, the following parameters of recent use were observed within the first hour after smoking: presence of CBN; presence of Δ⁹-THCV; presence of CBG; presence of CBC; presence of CBGA; and a very high ratio of Δ⁹-THC in breath compared to blood.

In the exhaled breath study, all 44 subjects self-reported peak impairment within the first 20 minutes after smoking (see Table 1), which coincided with the shortest cannabinoid half-lives (Table 5) and peak incidence of horizontal gaze nystagmus (FIGS. 5 and 6).

Evidence of recent cannabis use by inhalation through smoking or vaping dissipates rapidly in exhaled breath, and thus allowing 20 minutes between breath samplings, which was employed in the blood-based study and for most of the subjects in the blood and breath-based study, may result in the loss of valuable data. In the last nine subjects involved in the exhaled breath study, a back-to-back sample collection strategy was employed whereby two exhaled breath samples were collected in quick succession at 20 and 40 minutes post-smoking. Approximately two minutes elapsed between the collection of each sample. The very short half-lives observed for Δ⁹-THC, CBN, and other cannabinoids (approximately five minutes or less) in exhaled breath would allow for rapid sampling (see Table 6 below). The pharmacokinetic data from these nine subjects showed that this strategy is feasible. This approach could save a great deal of time and potentially result in more effective testing.

TABLE 6
Summary of cannabinoid half-lives in breath post-smoking:
back-to-back sampling strategy

| | Cannabinoid Half-Lives (min) 20 & 40 Minutes | | | | | | | | | |
|---|---|---|---|---|---|---|---|---|---|---|
| | Post-Smoking | | | | | | | | | |
| | Δ⁹-THC | | CBN | | CBC | | CBG | | Δ⁹-THC | |
| Subject | 20 | 40 | 20 | 40 | 20 | 40 | 20 | 40 | 20 | 40 |
| 89 | 1.2 | —* | 1.1 | — | 1.0 | — | 0.7 | — | 0.9 | — |
| 90 | 1.9 | 2.2 | 2.0 | 2.9 | 2.2 | 2.3 | 1.6 | — | 1.9 | — |
| 91 | 2.3 | 9.9 | 2.3 | 4.8 | 2.0 | 3.5 | 1.3 | — | 2.1 | — |
| 92 | 2.9 | — | 2.7 | — | 2.7 | — | 2.6 | — | 2.8 | — |
| 93 | 1.0 | 3.7 | — | — | — | — | — | — | — | — |
| 94 | 1.4 | —† | — | — | 1.3 | — | — | — | — | — |
| 95 | 2.1 | 5.9 | 1.8 | — | 2.0 | 5.2 | 2.1 | — | 1.6 | — |
| 96 | 7.3 | 8.6 | 6.4 | 10.7 | 11.8 | — | — | — | 3.8 | — |
| 97 | 11.8 | — | — | — | — | — | — | — | — | — |
| Average = | 3.5 | 6.1 | 2.7 | 6.1 | 3.3 | 3.6 | 1.7 | — | 2.2 | — |
| SD = | 3.6 | 3.2 | 1.9 | 4.1 | 3.8 | 1.5 | 0.7 | — | 1.0 | — |
| N = | 9 | 5 | 6 | 3 | 7 | 3 | 5 | — | 6 | — |

*Half-life not calculable.
†Outlier value (53.1) removed.

This Example demonstrated that the test method based on a combination of exhaled breath and blood sampling was 100% effective in identifying recent cannabis use within the three-hour impairment window, with no false positive test results observed. Just as observed in subject blood samples, multiple cannabinoids, including Δ⁹-THC, were detected in exhaled breath prior to smoking. Similar to the blood-based study, these subjects were instructed to abstain from cannabis use for at least 12 hours, but no more than 24 hours. This finding reinforces the fact that detecting Δ⁹-THC in the breath in and of itself does not constitute sufficient evidence of recent cannabis use or impairment.

### Example 6: Using a Single Breath Sample to Identify Subjects in the Peak Impairment Window after Smoking Cannabis

In the cannabis smoking study described in Example 5 above, a total of 34 of the 44 subjects had breath samples collected at baseline prior to smoking, all 44 subjects had breath samples collected up to one hour after smoking, and 40 of the 44 subjects had breath samples evaluated between one hour and three hours after smoking. Six (6) parameters were found to be associated with recent use of cannabis through inhalation within the peak impairment window: the presence of Δ⁹-THC, CBN, CBC, CBG, CBGA, and Δ⁹-THCV. Table 3 above summarizes the percent positivity for each of these parameters prior to smoking (baseline), within the peak impairment window (up to 60 minutes after smoking), and more than 60 minutes after smoking (outside of the peak impairment window).

The data in Table 3 above shows that these six cannabinoids are predominantly present in exhaled breath within the peak impairment window. Critically, two of these cannabinoids, CBC and Δ⁹-THCV were observed only within the peak impairment window. Based on this data, a test subject is positive for recent cannabis use by inhalation within the peak impairment window when they are positive for CBC and/or Δ⁹-THCV. For this study, 0/34 subjects (0%) tested positive at baseline prior to smoking, 39/40 subjects (97.5%) tested positive during the peak impairment window (≤60 minutes after smoking), and 0/40 subjects (0%) were tested positive outside of the peak impairment window (>60 minutes after smoking). Thus, no false positive test results were observed, and false negatives were minimal, making the single breath sample test a convenient, non-invasive, and accurate means of detecting a recent cannabis user who is still within the peak impairment window following use by inhalation.

The results from these six examples demonstrate that the described test method based on exhaled breath and blood sampling, or on exhaled breath sampling alone, can accurately determine whether a subject has used cannabis recently and whether the subject is also within the peak impairment window after use through inhalation (i.e., smoking or vaping). Unlike existing test methods that rely on single measures of cannabinoids in blood, urine, saliva, or breath, the described test can detect recent cannabis use within the peak impairment window without false positive test results in a background of non-recent cannabis use. This is a critical point because an effective test for recent cannabis use must be able to discriminate between past use and recent use, which carries the risk of impairment. It is the test method itself that makes this possible, not simply the use of blood and/or breath to detect cannabinoids. This test takes into account kinetic changes in cannabinoid levels between samples, changes in cannabinoid ratios between samples, as well as the presence of key recent use indicators, particularly in exhaled breath.

While the comprehensive breath and blood test is limited to detecting recent cannabis use only through inhalation within the impairment window, the described blood-based test could be applied to detecting recent use and impairment following oral consumption of cannabis products. Hypothetically, a similar strategy utilizing two blood samples could be deployed for detecting recent use of orally administered cannabis as well as other orally administered impairing drugs. It is well known that cannabinoid pharmacokinetics differ depending on the route of administration. Because Δ⁹-THC metabolites cannot be detected in breath, the blood may contain critical information pertaining to recent use, including concentrations of glucuronide metabolites and changes in the ratios of Δ⁹-THC metabolites such as 11-hydroxy-Δ⁹-THC and 11-nor-9-carboxy-Δ⁹-THC to Δ⁹-THC and to each other.

While the focus of this invention has been on the detection of recent inhaled cannabis use, it should be emphasized that a two-point breath and one-point blood test, for example, is not limited to just cannabis. The same testing strategy employed for cannabis may also prove to be useful for detecting recent use of other impairing drugs such as methamphetamine, phencyclidine, and cocaine that can be administered through vaporization. Exhaled breath testing has already been proven useful for detecting multiple drug types, and this test allows simultaneous testing for cannabis as well as other potentially impairing drugs in both breath and blood. Potential applications include sports medicine, enforcement of workplace drug policy, and law enforcement.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A method for determining recent use of cannabis within the impairment window in a subject, the method comprising the collection of one or more exhaled breath samples separated in time by approximately three minutes and one or more whole blood samples separated in time by approximately 20 minutes, analyzing them for Δ⁹-THC, Δ⁹-THC metabolites, and other cannabinoids, and then computing specific breath- and blood-based pharmacokinetic parameters and cannabinoid profiles to determine recent use of cannabis within the impairment window.

2. The method of claim 1, whereby the subject has used cannabis through smoking, vaping, or any other route of inhalation.

3. The method of claim 2, whereby a single blood sample can be used as a confirmatory test to support a finding of recent cannabis use within the impairment window based on two breath samples.

4. The method of claim 3, whereby the breath/blood Δ⁹-THC concentration or intensity ratio is calculated as the basis for confirmation.

5. The method of claim 2, whereby the subject is tested for recent cannabis use and impairment by an employer or prospective employer.

6. The method of claim 5, whereby the subject is tested for recent cannabis use and impairment by collecting at least one blood sample using a device that automatically collects and stores capillary blood for later laboratory analysis, and by collecting at least one breath sample using a device containing an electrostatic polymer filter that traps exhaled breath aerosols for later laboratory analysis.

7. The method of claim 5, whereby the subject is tested for recent cannabis use and impairment by collecting at least one blood sample using a lancet or other means of blood collection, and by collecting at least one breath sample using a device containing an electrostatic polymer filter.

8. The method of claim 5, whereby the subject is tested for recent cannabis use only by collecting two or more blood samples using a device that automatically collects and stores capillary blood for later laboratory analysis, or by collecting two or more blood samples using any other acceptable means of blood collection.

9. The method of claim 2, whereby the subject, as a result of suspicion of driving under the influence of cannabis, is tested for recent cannabis use and impairment by law enforcement personnel by collecting two exhaled breath samples and two blood samples.

10. The method of claim 9, whereby the blood samples are collected using a device that automatically collects and stores capillary blood for later laboratory analysis, and the breath samples are collected using a device containing an electrostatic polymer filter that traps exhaled breath aerosols for later laboratory analysis.

11. The method of claim 1, whereby the pharmacologic parameters used for determining recent use of cannabis are specifically adapted for oral administration of cannabis.

12. The method of claim 5, whereby the samples are analyzed and assessed using recent use parameters specific for oral consumption of cannabis.

13. The method of claim 6, whereby the samples are analyzed and assessed using recent use parameters specific for oral consumption of cannabis.

14. The method of claim 7, whereby the samples are analyzed and assessed using recent use parameters specific for oral consumption of cannabis.

15. The method of claim 8, whereby the samples are analyzed and assessed using recent use parameters specific for oral consumption of cannabis.

16. The method of claim 9, whereby the samples are analyzed and assessed using recent use parameters specific for oral consumption of cannabis.

17. The method of claim 10, whereby the samples are analyzed and assessed using recent use parameters specific for oral consumption of cannabis.

18. A method for determining recent use of drugs of abuse other than cannabis, the drugs of abuse consisting of natural and illegal synthetic cannabinoids, methamphetamine, opiates, benzodiazepines, barbiturates, cocaine, phencyclidine, psilocybin, and other potential drugs of abuse, the method comprising the collection of one or more whole blood samples separated in time by approximately 20 minutes and one or more exhaled breath samples separated in time by approximately three minutes, analyzing them for key drug molecules and metabolites, computing the changes in specific pharmacokinetic parameters and intensity ratios between samples, and then evaluating the results based on the criteria of one or more breath-based and blood-based parameters associated with the recent use of each respective drug compound.

19. The method of claim 18, whereby the subject is tested for recent drug use by an employer or prospective employer.

20. The method of claim 19, whereby the subject is tested for recent drug use by collecting at least one blood sample using a device that automatically collects and stores capillary blood for later laboratory analysis, and by collecting at least one breath sample using a device containing an electrostatic polymer filter that traps exhaled breath aerosols for later laboratory analysis.

21. The method of claim 19, whereby the subject is tested for recent drug use by collecting at least one blood sample using a lancet or other means of blood collection, and by collecting at least one breath sample using a device containing an electrostatic polymer filter.

22. The method of claim 19, whereby the subject is tested for recent drug use by collecting at least one sample of oral fluid or urine in addition to blood and exhaled breath.

23. The method of claim 18, whereby the subject, as a result of suspicion of driving under the influence of drugs, is tested for recent drug use by law enforcement personnel by collecting two exhaled breath samples and two blood samples.

24. The method of claim 23, whereby the blood samples are collected using a device that automatically collects and stores capillary blood for later laboratory analysis, and the breath samples are collected using a device containing an electrostatic polymer filter that traps exhaled breath aerosols for later laboratory analysis.
